(19) United States
(12) Patent Application Publication
(10) Pub. No.: US 2010/0298726 A1
(43) Pub. Date: Nov. 25, 2010

(54) LOW-PRESSURIZATION BLOOD PRESSURE MONITORING APPARATUS AND METHOD

(75) Inventors: Seok Chan KIM, Seoul (KR); Jong-pal KIM, Seoul (KR); Youn-ho KIM, Hwaseong-si (KR)

Correspondence Address: CANTOR COLBURN LLP, 20 Church Street, 22nd Floor, Hartford, CT, 06103, US

(73) Assignee: SAMSUNG ELECTRONICS CO., LTD., Suwon-si (KR)

(21) Appl. No.: 12/778,415

(22) Filed: May 12, 2010

(30) Foreign Application Priority Data
May 22, 2009 (KR) 10-2009-0045203

(51) Int. Cl. A61B 5/0225

(52) U.S. Cl. 600/493

(57) ABSTRACT

A blood pressure monitoring apparatus including a pressing unit which presses a measurement body part of a subject, a sensing unit which senses a sphygmus wave at the measurement body part while the measurement body part is being pressed, a control unit controlling a point of time of stopping the pressing based on an amplitude of the sensed sphygmus wave, and an estimation unit which estimates a blood pressure of the subject based on the sphygmus wave sensed before the pressing performed by the pressing unit is stopped.

### CROSS-REFERENCE TO RELATED APPLICATIONS

This application claims priority to Korean Patent Application No. 10-2009-0045203, filed on May 22, 2009, and all the benefits accruing therefrom under 35 U.S.C. §119, the disclosure of which is incorporated herein in its entirety by reference.

### BACKGROUND

1. Field

Provided is a low-pressurization blood pressure monitoring apparatus and method.

2. Description of the Related Art

Blood pressure is used as an index of a person's health condition. Apparatuses for measuring blood pressure are commonly used in medical institutions and at home. The United States Food and Drug Administration (“FDA”) requires the standards for apparatuses for measuring blood pressure to comply with the requirements of the Association for the Advancement of Medical Instrumentation (“AAMI”). The American National Standards Institute (“ANSI”)/AAMI SP10 issued by the AAMI offers specification details, and safety and performance requirements for the apparatuses.

### SUMMARY

Provided is a blood pressure monitoring apparatus and method in which a user is allowed to select a pressurization mode, so that the user may conveniently measure blood pressure with less pain by selecting a low-pressurization mode in which blood vessels are not occluded.

Provided is a computer readable recording medium having recorded thereon a computer program for executing the method.

Provided is a blood pressure monitoring apparatus including a pressing unit that presses a measurement body part of a subject, a sensing unit that senses a sphygmus wave at the measurement body part, a control unit that stops the pressing performed by the pressing unit based on an amplitude of the sensed sphygmus wave, and an estimation unit that estimates blood pressure of the subject based on the sphygmus wave sensed before the pressing performed by the pressing unit is stopped.

Provided is a blood pressure monitoring method including monitoring an amplitude of a sphygmus wave sensed while a measurement body part of a subject is being pressed, controlling a point of time of stopping the pressing according to a result of the monitoring so that the pressing is stopped after the amplitude of the sensed sphygmus wave has reached a maximum, and estimating blood pressure of the subject based on the sphygmus wave sensed before the pressing performed by the pressing unit is stopped.

Provided is a computer readable recording medium having embodied thereon a program for executing the blood pressure monitoring method.

### BRIEF DESCRIPTION OF THE DRAWINGS

These and/or other aspects will become apparent and more readily appreciated from the following description of the examples, taken in conjunction with the accompanying drawings of which:

FIG. 1 is a diagram illustrating an exemplary embodiment of a configuration of a blood pressure monitoring apparatus;

FIG. 2 is a diagram for describing exemplary embodiments of waveforms filtered by a filtering unit;

FIG. 3 is a graph for describing an exemplary embodiment of a method of monitoring an amplitude of a sphygmus wave;

FIG. 4 is a detailed diagram of an exemplary embodiment of a blood pressure operation unit illustrated in FIG. 1;

FIG. 5 is a graph of an exemplary embodiment of a sphygmus wave detected at a measurement body part of a subject, when a full-pressurization mode is selected;

FIG. 6 is a graph of an exemplary embodiment of a sphygmus wave detected at a measurement body part of a subject, when a low-pressurization mode is selected;

FIG. 7 is a flowchart of an exemplary embodiment of a low-pressurization blood pressure monitoring method;

FIG. 8 is a flowchart of an exemplary embodiment of a blood pressure monitoring method including selecting a pressurization mode; and

FIG. 9 is a flowchart of an exemplary embodiment of a method of correcting a relational equation.

### DETAILED DESCRIPTION

The invention is described more fully hereinafter with reference to the accompanying drawings, in which exemplary embodiments of the invention are shown. This invention may, however, be embodied in many different forms and should not be construed as limited to the exemplary embodiments set forth herein. Rather, these embodiments are provided so that this disclosure will be thorough and complete, and will fully convey the scope of the invention to those skilled in the art.

It will be understood that when an element or layer is referred to as being “connected to” another element or layer, the element or layer can be directly connected to another element or layer or intervening elements or layers. In contrast, when an element is referred to as being “directly connected to” another element or layer, there are no intervening elements or layers present. As used herein, connected may refer to elements being physically and/or electrically connected to each other. Like numbers refer to like elements throughout. As used herein, the term “and/or” includes any and all combinations of one or more of the associated listed items.

It will be understood that, although the terms first, second, third, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

Hereinafter, the invention will be described in detail with reference to the accompanying drawings.

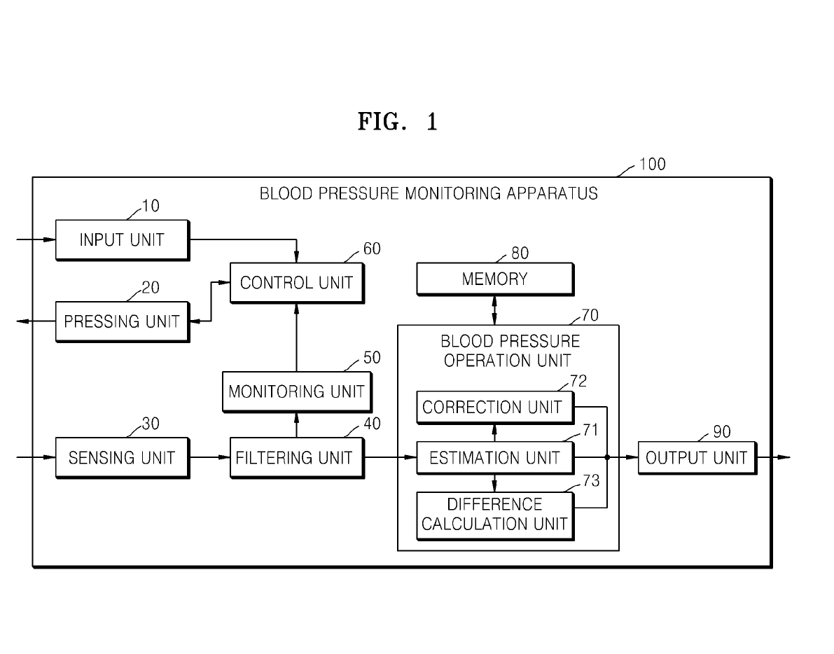

FIG. 1 is a diagram illustrating an exemplary embodiment of a configuration of a blood pressure monitoring apparatus 100. Referring to FIG. 1, the blood pressure monitoring apparatus 100 includes an input unit 10, a pressing unit 20, a sensing unit 30, a filtering unit 40, a monitoring unit 50, a control unit 60, a blood pressure operation unit 70, a memory 80, and an output unit 90. In the present specification, only components that are relevant to the present disclosure will be described in order to prevent making the features of the present disclosure vague. However, it will be understood by one of ordinary skill in the art that any other general-use components may also be included in addition to the components illustrated in FIG. 1.

The blood pressure monitoring apparatus 100 is an apparatus in which blood pressure is measured while a measurement body part of a subject is being pressed, where a user may select a degree of pressing the measurement body part, e.g., a low-pressurization mode or a full-pressurization mode, in order to measure blood pressure at the measurement body part of the subject. In one exemplary embodiment, the user may measure blood pressure at a measurement body part of a subject, while the measurement body part of the subject is being pressed so as to occlude or not to occlude a blood vessel in the measurement body part of the subject. Hereinafter, for convenience of explanation, where the measurement body part is pressed so as to occlude the blood vessel it will be referred to as “full-pressurization mode”, and where the measurement body part is pressed so as not to occlude the blood vessels it will be referred to as “low-pressurization mode.” It will be understood by those of ordinary skill in the art that the measurement body part of the subject may include any body part for measuring blood pressure, such as a wrist, a finger, an upper arm, or the like.

The blood pressure is pressure on the walls of blood vessels as blood pumped out of the heart flows along the blood vessels, and includes arterial blood pressure, capillary blood pressure, and venous blood pressure, according to the blood vessels where blood pressure is measured. The blood pressure varies according to heartbeats. Also, the blood pressure includes systolic blood pressure when blood flows into the arteries as the ventricles of the heart contract, and diastolic blood pressure on the arterial walls due to the elasticity of the arterial walls even when the ventricles expand and blood stays in the ventricles.

A sphygmus wave is a wave generated as a pulse reaches peripheral nerves. The pulse is a phenomenon whereby the pressure of blood flowing into the aorta due to heartbeats, affects other arteries. That is, whenever the heart contracts, blood is provided from the heart to every part of the human body through the aorta, and the pressure on the aorta varies. The variation in pressure is transferred to peripheral arterioles of the hands and feet. The sphygmus wave represents the variation in pressure as a waveform.

It will be understood by one of ordinary skill in the art that the blood pressure monitoring apparatus 100 in FIG. 1 may measure at least one selected from the group consisting of the sphygmus wave and the pressure on the walls of the blood vessels, in order to measure the blood pressure. Hereinafter, for convenience of explanation, a blood pressure monitoring method is a method of measuring at least one selected from the group consisting of blood pressure and a sphygmus wave, as selected by the user.

The blood pressure monitoring apparatus 100 may measure blood pressure by using a noninvasive method. The noninvasive method measures blood pressure while a body part at which blood pressure is to be measured is being pressed. In one exemplary embodiment of a noninvasive method, blood pressure is measured when the bloodstream in the aorta, the brachial artery or the radial artery is occluded by winding a blood-pressure cuff around a measurement body part of a subject, and then pressurizing the measurement body area by injecting air into the blood-pressure cuff.

In the noninvasive method, the blood pressure is measured from outside the blood vessels. Examples of the noninvasive method include, but are not limited to, an auscultatory method of measuring blood pressure using Korotkoff sounds, an oscillometric method of measuring blood pressure using oscillations generated due to the flow of blood, a tonometric method using a tonometer, and a method using pulse transit time (“PTT”).

With regard to the noninvasive method, in the auscultatory method, a body part where arterial blood flows is pressed sufficiently to stop the flow of arterial blood and then is released, pressure at a moment when an initial pulse is heard is measured as the systolic blood pressure, and pressure at a moment when no more pulse is heard is measured as the diastolic blood pressure.

The oscillometric method and the tonometric method are used in digital blood pressure measuring apparatuses. Like the auscultatory method, in the oscillometric method, the systolic blood pressure and the diastolic blood pressure are measured by sensing oscillations of blood vessels, which are generated when a body part is pressed sufficiently to stop the flow of arterial blood and then is released. Pressure at regular ratios of amplitude with respect to a maximum amplitude of the oscillations of the blood vessels may be measured as the systolic blood pressure and the diastolic blood pressure.

When measuring blood pressure by using the noninvasive method, the measurement body part of the subject is pressed until the flow of blood stops, e.g., until the blood vessel is occluded. For a subject who periodically and frequently measures blood pressure, such repeated occlusion of the blood vessel impedes the flow of blood so that blood pressure of the subject may be inaccurately measured or side effects may be caused due to the insufficient supply of blood to peripheral body parts. In addition, the subject may feel pain with strong pressurization or may be bruised due to the rupture of capillary blood vessels.

It is convenient for a hypertension patient that a higher pressure than a normal pressure level is applied. However, the blood pressure monitoring apparatus 100 of the invention may measure blood pressure in a low-pressurization mode so as not to inconvenience the subject. In addition, due to a shorter pressurizing duration, the overall blood pressure measurement duration may be reduced. The blood pressure monitoring apparatus 100 allows the user to select a pressurization mode such that a patient does not always have to use an inconvenient pressure. In an exemplary embodiment, blood pressure may be periodically measured in the full-pressurization mode, a relational equation that is used to estimate blood pressure in the low-pressurization mode may be corrected by using the measured blood pressures in the full-pressurization mode, and a difference between the blood pressure measured in the low-pressurization mode and the blood pressure measured in the full-pressurization mode may be calculated.

It will be understood by one of ordinary skill in the art that the blood pressure monitoring apparatus 100 may be applied to all blood pressure measuring methods using a noninvasive method, and may be used in, for example, an upper arm-type, wrist-type or finger-type hemadynamometer. The blood pressure monitoring apparatus 100 reduces the amount of pain suffered by the subject and reduces the blood pressure measurement duration.

Referring again to FIG. 1, the input unit 10 obtains an input signal from the user. The input signal is for selecting a pressurization mode that determines a degree of pressing by the pressing unit 20. The user, via the input unit 10, may select a pressurization mode that determines the degree of pressing by the pressing unit 20. The pressurization mode may include at least one selected from the group consisting of the full-pressurization mode in which a measurement body part of a subject is pressurized until the blood vessel in the measurement body part is occluded, and the low-pressurization mode in which the measurement body part is pressurized so as not to occlude the blood vessel. The input unit 10 may include any apparatus for obtaining the input signal from the user. In exemplary embodiments, the input unit 10 may include a keyboard, a mouse, a touch pad, a speech recognition apparatus, or the like.

The pressing unit 20 presses a measurement body part at which blood pressure is to be measured. The pressing unit 20 may include a pressurizer, for example, a cuff or a wrist band, etc., for pressing the measurement body part, and an actuator for driving the pressurizer to expand or contract. The measurement body part includes any body part which has a blood vessel and at which blood pressure is measurable by using the above-described blood pressure measuring methods, such as an upper arm having the brachial artery, or a wrist having the radial artery. The pressing unit 20 allows the pressurizer to expand or contract by using the actuator so as to press the measurement body part at which blood pressure is to be measured, such as an upper arm, a wrist, or a finger. Also, a point of time at which the pressing unit 20 stops pressurizing may be controlled by the control unit 60.

The sensing unit 30 sense pressure and a sphygmus wave in a blood vessel in the pressed measurement body part, by using at least one sensor while the measurement body part is being pressed. Although a pressure sensor, a photoplethysmography (“PPG”) sensor, etc. may be generally used as the sensor, the sensing unit 30 is not limited thereto. In an exemplary embodiment, the sensor may be any apparatus for obtaining pressure values in a blood vessel. That is, the sensing unit 30 senses the pressure and the sphygmus wave in the blood vessel of the body part pressed by the pressing unit 20, by using the sensor.

In more detail, the pressing unit 20 gradually increases the pressure applied to pressurize the measurement body part of the subject and stops pressing the measurement body part under the control of the control unit 60 according to the pressurization mode selected by the user. If the pressurization mode selected by the user is the full-pressurization mode, the pressing unit 20 presses the measurement body part until the flow of arterial blood stops and then stops pressing. In general, the amount of applied pressure at which the flow of arterial blood stops is greater than or equal to a systolic blood pressure. In one exemplary embodiment, if the applied pressure reaches 140 millimeters of mercury (mmHg) while the pressing unit 20 gradually increases the pressure applied to press the measurement body part, the pressing unit 20 stops pressing. However, the pressure applied to occlude the blood vessel of the subject is determined to be 140 mmHg as an example, and the amount of pressure may be varied according to an usage environment.

If the pressurization mode selected by the user is the low-pressurization mode, the pressing unit 20 stops pressing under the control of the control unit 60. A method of controlling the point of time at which the pressing unit 20 stops pressing when the low-pressurization mode is selected, will be described below in connection with the monitoring unit 50.

The sensing unit 30 senses pressure and a sphygmus wave in the blood vessel of the pressed body part, for example, the sensing unit 30 measures the pressure and the sphygmus wave in the blood vessel of the pressed body part for a period of time, from before or when the pressing unit 20 presses the part until after the pressing unit 20 stops pressing the body part. The period of time for sensing may be arbitrarily set by the user, and may be generally set to be a period from when arterial blood stops flowing until when arterial blood normally circulates, when the full-pressurization mode has been selected. The sensing unit 30 senses the pressure in the blood vessel and transmits the obtained pressure values to the filtering unit 40.

The filtering unit 40 separately passes a high-frequency component and a low-frequency component of the pressure values obtained by the sensing unit 30. The filtering unit 40 includes a high-pass filter that passes a higher-frequency signal than a cutoff frequency without attenuation and attenuates a lower-frequency signal than the cutoff frequency, and a low-pass filter that passes a lower-frequency signal than the cutoff frequency without attenuation and attenuates a higher-frequency signal than the cutoff frequency.

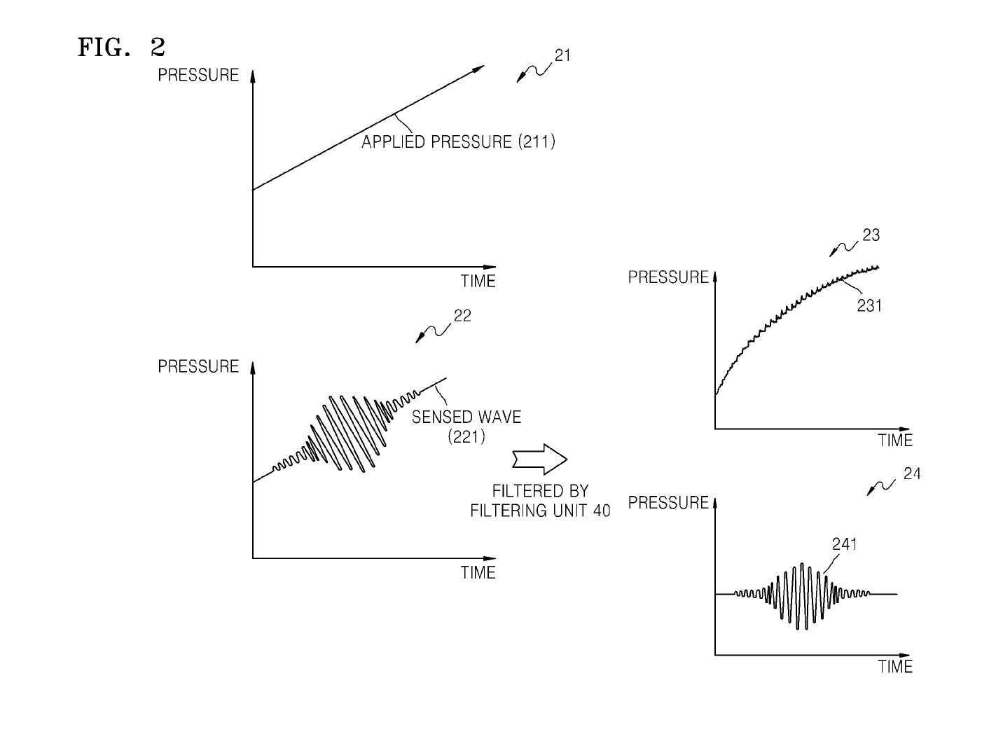

The filtering unit 40 will be described in detail. FIG. 2 is a diagram for describing exemplary embodiments of waveforms filtered by the filtering unit 40 in FIG. 1. Referring to FIG. 2, a graph 21 of pressure applied to press a measurement body part, a graph 22 of a pressure wave sensed at the measurement body part, and graphs 23 and 24 of the waveforms filtered by the filtering unit 40, are illustrated.

The graph 21 of applied pressure illustrates a condition for increasing the pressure applied to the measurement body part at which blood pressure is measured by the pressing unit 20. As described above, applied pressure 211, which is applied by the pressing unit 20, is continuously increased and then is released. The control unit 60 controls the point of time at which the pressing unit 20 stops pressing, according to the pressurization mode selected by using the input unit 10. In one exemplary embodiment, if the full-pressurization mode is selected, the applied pressure 211 may be increased to 140 mmHg, and then the pressing unit 20 stops pressing. If the low-pressurization mode is selected, the pressing unit 20 may stop pressing, e.g., applying the applied pressure 211, at a point of time when the amplitude of the sphygmus wave decreases for the first time, according to a result of monitoring the amplitude of the sphygmus wave by the monitoring unit 50.

The graph 22 illustrates a wave 221 sensed by the sensing unit 30, e.g., a sphygmus wave and a pressure wave in the blood vessel of the measurement body part. The wave 221 sensed by the sensing unit 30 includes both a high-frequency component and a low-frequency component. The high-pass filter of the filtering unit 40 passes a high-frequency component signal (for example, a signal having a frequency of 0.5 hertz (Hz) to 30 hertz (Hz) and attenuates a low-frequency component signal. The low-frequency filter of the filtering unit 40 passes the low-frequency component signal (for example, a signal having a frequency of less than 0.5 Hz) and attenuates the high-frequency component signal. Thus, pressure values obtained by the sensing unit 30 are filtered by the filtering unit 40 so as to form a filtered waveform 231 having the low-frequency component and a filtered waveform 241 having the high-frequency component.

The blood pressure monitoring apparatus 100 in FIG. 1 uses the high-frequency component signal, and thus, hereinafter, for convenience of explanation, the waveform filtered by the filtering unit 40 will refer to the waveform 241 having the high-frequency component. The filtering unit 40 may include a general high-pass filter and low-pass filter, which are well known to one of ordinary skill in the art, and thus a detailed description thereof will not be provided here.

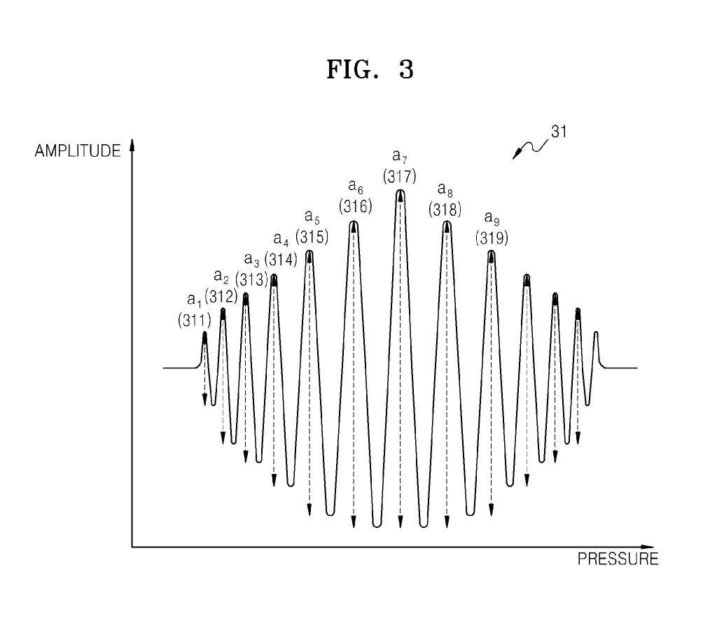

Referring back to FIG. 1, the monitoring unit 50 monitors the amplitude of the signal filtered by the filtering unit 40. In detail, the monitoring unit 50 monitors the amplitude of the high-frequency component waveform among the waveforms filtered by the filtering unit 40, and transmits the results of the monitoring to the control unit 60. FIG. 3 is a graph for describing an exemplary embodiment of a method of monitoring the amplitude of a sphygmus wave.

Referring to FIG. 3, a pressure waveform 31 having the high-frequency component is plotted as a relation of amplitude with respect to pressure. The pressure waveform 31 having the high-frequency component may correspond to the waveform 241 filtered by the high-pass filter illustrated in FIG. 2. As illustrated in FIG. 3, assuming that amplitudes are denoted by a₁ (311), a₂ (312), a₃ (313), and so on, the monitoring unit 50 monitors the amplitudes a₁ (311), a₂ (312), a₃ (313), and so on, and transmits the result of the monitoring to the control unit 60.

In one exemplary embodiment, if the low-pressurization mode is selected, and an event where the amplitude that has increased starts to decrease for the first time is detected, the monitoring unit 50 transmits the result of the monitoring to the control unit 60. Alternatively, if the full-pressurization mode is selected, and the amplitude corresponding to a pressure at which the blood vessel is occluded is detected during monitoring, the monitoring unit 50 transmits the result of the monitoring to the control unit 60. Hereinafter, an exemplary embodiment of a method of controlling the pressing unit 20 by the control unit 60 will be described in detail.

Referring back to FIG. 1, the control unit 60 controls the pressing unit 20 to stop pressing based on the amplitude of the sensed sphygmus wave. The control unit 60 controls the pressing unit 20 by obtaining the input signal from the input unit 10 and the result of monitoring of amplitude from the monitoring unit 50. As described in connection with the input unit 10, when the input signal that selects one of the full-pressurization mode or the low-pressurization mode is obtained from the input unit 10, the control unit 60 controls the pressing unit 20 to stop pressing based on the result of monitoring obtained from the monitoring unit 50.

In one exemplary embodiment, if the full-pressurization mode is selected, the control unit 60 may control the pressing unit 20 to stop pressing when the amplitude, which is obtained from the monitoring unit 50 as the result of monitoring, reaches a level used to estimate blood pressure or when the pressure applied by the pressing unit 20 reaches a pressure at which the blood vessel is occluded.

As described above, if the full-pressurization mode is selected, the measurement body part of the subject is pressed until the blood vessel is occluded. Thus, when the applied pressure gradually increases and reaches 140 mmHg, known as a pressure level at which the blood vessel is occluded, the control unit 60 stops the pressing performed by the pressing unit 20. Referring to FIG. 2, when the applied pressure 211 gradually increases and reaches an arbitrary pressure, for example, 140 mmHg, the pressing is stopped at that point of time.

In the case where the control unit 60 controls the pressing unit 20 by using the result of monitoring obtained from the monitoring unit 50, the control unit 60 may stop the pressing performed by the pressing unit 20 when the amplitude of the sphygmus wave has a predetermined ratio at which blood pressure is to be estimated, with respect to the maximum amplitude of the sphygmus wave. In one exemplary embodiment, referring to FIG. 3, the monitoring unit 50 monitors the amplitude of the filtered waveform to detect an instant that the amplitude of the filtered waveform reaches the maximum a₇ (317), and an instant that the amplitude of the filtered waveform reaches a ratio at which blood pressure is to be estimated, with respect to the maximum amplitude a₇ (317).

The ratio at which blood pressure is to be estimated refers to a characteristic ratio that is commonly used to estimate systolic blood pressure, and may be, for example, about 40% of the maximum amplitude. However, the ratio at which blood pressure is to be estimated is not limited thereto and may vary, as will be understood by one of ordinary skill in the art. If the ratio at which blood pressure is to be estimated is about 40%, the monitoring unit 50 monitors the amplitude of the filtered waveform to detect an instant that the amplitude of the filtered waveform reaches about 40% of the maximum amplitude a₇ (317), and transmits the detected result to the control unit 60. Then, the control unit 60 may stop the pressing performed by the pressing unit 20. In the illustrated embodiment, the point of time at which the pressing stops corresponds to an instant that an amplitude of the filtered waveform that is smaller than about 40% of the maximum amplitude is detected.

In one exemplary embodiment, if the low-pressurization mode is selected, the control unit 60 stops the pressing performed by the pressing unit 20 after the amplitude of the sensed sphygmus wave has reached the maximum. In other words, the control unit 60 may stop the pressing performed by the pressing unit 20 at an instant when the amplitude of the filtered waveform starts to decrease for the first time, as a result of the monitoring performed by the monitoring unit 50. The control unit 60 may stop the pressing performed by the pressing unit 20 at an instant when the amplitude of the sphygmus wave that has continuously increased starts to decrease for the first time, as a result of the monitoring performed by the monitoring unit 50.

Referring to FIG. 3, at an instant when the monitoring unit 50 detects that the amplitude of the filtered waveform decreases to a₈ (318) from the maximum amplitude a₇ (317), the control unit 60 stops the pressing performed by the pressing unit 20. However, stopping the pressing performed by the pressing unit 20, when blood pressure of the subject is measured in the low-pressurization mode, at an instant when the amplitude of the sphygmus wave starts to decrease for the first time is an example, and thus it will be understood by one of ordinary skill in the art that the pressing may be stopped at any time after the amplitude of the sensed sphygmus wave has reached the maximum, without limitation.

As described above, when the low-pressurization mode is selected, blood pressure of the subject may be measured while the pressing unit 20 presses the measurement body part of the subject so as not to occlude the blood vessel in the measurement body part. Thus, the subject may measure blood pressure without pain or side effects caused due to the occlusion of the blood vessel. In addition, due to the shorter pressurizing duration, it takes less time to measure blood pressure. This enables continuous blood pressure measurement.

Referring back to FIG. 1, the control unit 60 may correspond to one or a plurality of processors of the blood pressure monitoring apparatus 100. A processor may be realized by using an array of a plurality of logic gates, or a combination of a general-use microprocessor and memory for storing a computer program to be executed in the microprocessor. Also, it will be understood by one of ordinary skill in the art that the processor may be realized by using a different type of hardware. In addition to the pressing unit 20, the control unit 60 may also control the other components of the blood pressure monitoring apparatus 100.

The blood pressure operation unit 70 estimates blood pressure of the subject by using the waveform obtained from the filtering unit 40, corrects a relational equation for calculating systolic blood pressure by using the sphygmus wave measured in the full-pressurization mode, and a difference between the blood pressure measured in the low-pressurization mode and the blood pressure measured in the full-pressurization mode.

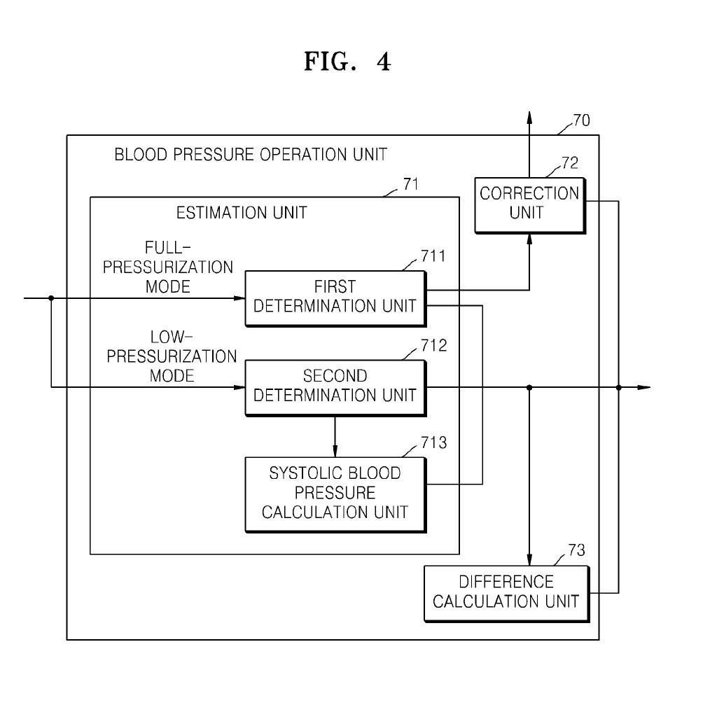

FIG. 4 is a detailed diagram of an exemplary embodiment of the blood pressure operation unit 70 illustrated in FIG. 1. Referring to FIG. 4, the blood pressure operation unit 70 includes an estimation unit 71, a correction unit 72, and a difference calculation unit 73. The estimation unit 71 includes a first determination unit 711, a second determination unit 712, and a systolic blood pressure calculation unit 713.

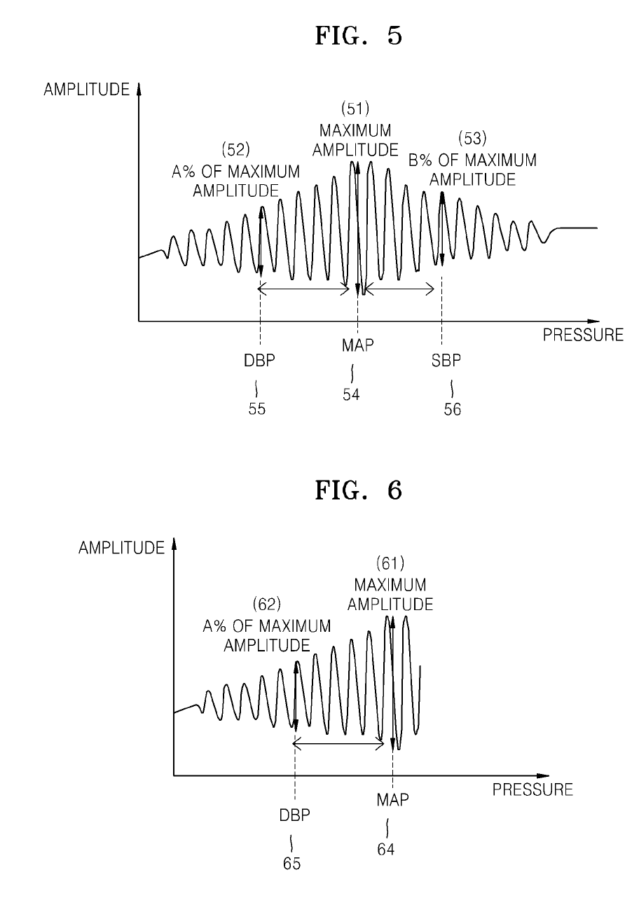

If the full-pressurization mode is selected, the first determination unit 711 determines at least one selected from the group consisting of systolic blood pressure, diastolic blood pressure and mean arterial pressure of the subject, by using the waveform obtained from the filtering unit 40. FIG. 5 is a graph of an exemplary embodiment of a sphygmus wave measured at a measurement body part of a subject when the full-pressurization mode is selected. Although the method will now be described with respect to an oscillometric method as a blood pressure determination method, as described above, the blood pressure measurement determination method is not limited to the oscillometric method. Referring to FIG. 5, the graph illustrates the filtered waveform 241 having the high-frequency component illustrated in FIG. 2, as a graph of amplitude with respect to pressure.

A pressure at a point where a sphygmus wave has a maximum amplitude 51 is referred to as mean arterial pressure (“MAP”) 54. The MAP 54 corresponds to pressure at a point of time when pressure applied by the pressing unit 20 is equal to pressure of the blood vessel sensed by the sensing unit 30. In a general blood pressure monitoring apparatus, diastolic blood pressure (“DBP”) 55 and systolic blood pressure (“SBP”) 56 are determined by using a characteristic ratio with reference to the MAP 54 of the sphygmus wave.

A pressure having a specific ratio of amplitude, which is used to estimate blood pressure, with respect to the maximum amplitude 51 of the sphygmus wave may be defined as the DBP 55 or the SBP 56, whereby the ratio used to estimate blood pressure may be defined as a characteristic ratio. Here, the estimated blood pressure may include at least one selected from the group consisting of the SBP 56 and the DBP 55. That is, pressure having an amplitude 52 of about A % with respect to the maximum amplitude 51 may be defined as the DBP 55, and pressure having an amplitude 53 of about B % with respect to the maximum amplitude 51 may be defined as the SPB 56.

The characteristic ratios such as A and B may vary according to a usage environment determined by a manufacturer or a user of the blood pressure monitoring apparatus 100 illustrated in FIG. 1. In one exemplary embodiment, the ratios A and B, which are used to estimate blood pressure, may be respectively set as about 70% and about 40%. That is, referring to FIG. 5, pressure having an amplitude of about 70% with respect to the maximum amplitude 41 may be defined as the DBP 55, and pressure having an amplitude of about 40% with respect to the maximum amplitude 41 may be defined as the SBP 56.

Referring back to FIG. 4, the first determination unit 711 determines at least one selected from the group consisting of the MAP 54, the DBP 55 and the SBP 56 by applying the characteristic ratio to the waveform obtained from the filtering unit 40. A method of determining the MAP 54, the DBP 55 and the SBP 56 has been described above with reference to FIG. 5, and thus a detailed description thereof will not be provided here. The blood pressures determined by the first determination unit 711 are estimated as the blood pressures of the subject measured in the full-pressurization mode and are then output to the user via the output unit 90 (FIG. 1). Alternatively, the blood pressures determined by the first determination unit 711 may be used by the correction unit 72 (FIG. 4) to update the relational equation stored in the memory 80, or may be used by the difference calculation unit 73 to calculate a difference between the blood pressure measured in the full-pressurization mode and the blood pressure measured in the low-pressurization mode.

If the low-pressurization mode is selected, the second determination unit 712 determines at least one selected from the group consisting of a DBP and an MAP of the subject by using the waveform obtained from the filtering unit 40. Since the low-pressurization mode has been selected, the second determination unit 712 obtains a waveform until the amplitude of the sphygmus wave has reached the maximum, from the filtering unit 40.

FIG. 6 is a graph of an exemplary embodiment of a sphygmus wave detected at a measurement body part of a subject when the low-pressurization mode is selected. The second determination unit 712 determines the MAP 64 and the DBP 65 by using the waveform obtained from the filtering unit 40. Since the low-pressurization mode has been selected, the second determination unit 712 obtains a waveform including one more amplitude from the instant when the amplitude of the sphygmus wave has reached the maximum 61, from the filtering unit 40. When the monitoring unit 50 detects an event when the amplitude of the sphygmus wave starts to decrease while monitoring the amplitude of the sphygmus wave, the monitoring unit 50 transmits the detected result to the control unit 60. The control unit 60 stops pressing by the pressing unit 20. Thus, the second determination unit 712 may obtain the waveform including the instant when the amplitude of the sphygmus wave has reached the maximum 61, and may determine at least one selected from the group consisting of the MAP 64 and the DBP 65 by using the obtained waveform. Since the blood vessel of the subject is not occluded in the low-pressurization mode, the second determination unit 712 does not determine systolic blood pressure.

Referring back to FIG. 4, the systolic blood pressure calculation unit 713 calculates systolic blood pressure based on the MAP and the DBP obtained from the second determination unit 712, since the second determination unit 712 does not determine systolic blood pressure. In detail, the systolic blood pressure calculation unit 713 may calculate the SBP by using a relational equation representing correlations between the MAP, the DBP and the SBP, the relational equation being stored in the blood pressure monitoring apparatus 100. It will be understood by one of ordinary skill in the art that the relational equation is previously stored in the memory 80 of the blood pressure monitoring apparatus 100, but is not limited thereto. In one exemplary embodiment, the relational equation may be obtained outside the blood pressure monitoring apparatus 100, or from the other units in the blood pressure monitoring apparatus 100, and then used to calculate systolic blood pressure.

An exemplary embodiment of a method of calculating systolic blood pressure by using the relational equation will now be described in detail. When blood pressure is measured at the brachial artery, about one third of a pressure wave in the brachial artery represents a systolic pressure wave, and the other two thirds of the pressure wave represent a diastolic pressure wave. This will be obvious to one of ordinary skill in the art, and thus a detailed description thereof will not be provided here. An equation of calculating mean arterial pressure by using diastolic and systolic blood pressures may be defined as Equation 1, based on the above-described general characteristics of the systolic and diastolic blood pressure waves generated as the heart contracts and relaxes pressure.

$$MAP = \frac{2}{3} DBP + \frac{1}{3} SBP \qquad [ Equation 1 ]$$

In Equation 1, MAP denotes the mean arterial pressure, DBP denotes the diastolic blood pressure, and SBP denotes the systolic blood pressure. Equation 1 may be rearranged as an equation for calculating the systolic blood pressure from the mean arterial pressure and the diastolic blood pressure, as defined in Equation 2.

$$SBP = 3 MAP - 2 DBP \qquad [ Equation 2 ]$$

In Equation 2, MAP denotes the mean arterial pressure, DBP denotes the diastolic blood pressure, and SBP denotes the systolic blood pressure. As defined in Equation 2, the SBP may be calculated by using the MAP and the DBP. Equation 2 is merely an exemplary relational equation representing the correlation between the MAP, the DBP and the SBP. Thus, it will be understood by one of ordinary skill in the art that the relational equation representing the correlation between the MAP, the DBP and the SBP is not limited to Equation 2.

Referring again to FIG. 4, the systolic blood pressure calculation unit 713 calculates the systolic blood pressure by substituting the MAP and the DBP obtained from the second determination unit 712 into the relational equation representing the correlation between the MAP, the DBP and the SBP. The MAP and the DBP determined by the second determination unit 712 and the SBP calculated by the systolic blood pressure calculation unit 713 may be displayed to the user via the output unit 90 (FIG. 1), or may be transmitted to the difference calculation unit 73 and used to calculate a difference between the blood pressure measured in the full-pressurization mode and the blood pressure measured in the low-pressurization mode.

The correction unit 72 corrects the relational equation representing the correlation between the MAP, the DBP and the SBP by using at least one selected from the group consisting of the MAP, the DBP and the SBP which are determined by the first determination unit 711. The relational equation may be defined as Equation 2, as described above.

However, the relational equation may be corrected according to the characteristics of the subject and then the relational equation previously stored in the memory 80 may be updated. Although the relational equation defined as Equation 2 is a generally used one, it does not reflect the physical characteristics of subjects or the characteristics of measurement body parts at which blood pressure is measured. Thus, the relational equation may be corrected by using data measured at the body of the subject in the full-pressurization mode, and blood pressure may be accurately measured in the low-pressurization mode by using the corrected relational equation. The relational equation may be corrected periodically, for example, weekly or monthly.

An exemplary embodiment of a method of correcting the relational equation will now be described in detail. A relational equation defined as Equation 2 may be defined as Equation 3 for correcting the relational equation.

SBP=nMAP−mDBP [Equation 3]

In Equation 3, SBP denotes the systolic blood pressure, MAP denotes the mean arterial pressure, DBP denotes the diastolic blood pressure, and n and m are positive numbers. In other words, Equation 2 may be corrected by using new n and m. Equation 2 is a default relational equation previously stored in the blood pressure monitoring apparatus 100, and is equivalent to Equation 3 where n and m are defined as 3 and 2, respectively. Thus, correcting the relational equation is to update the relational equation with n and m derived by using the blood pressure measured in the full-pressurization mode.

An exemplary embodiment of a method of deriving n and m will now be described in detail. As described above, when blood pressure is measured at the brachial artery, about one third of a pressure wave in the brachial artery represents a systolic pressure wave, and the other two thirds of the pressure wave represent a diastolic pressure wave. Herein, one third (⅓) and two thirds (⅔) of the pressure wave are fixed numbers not reflecting the characteristics of the measurement body part at which blood pressure is measured. Thus, the correlation between the diastolic blood pressure, the systolic blood pressure and the mean arterial pressure may be defined as Equation 4.

MAP=aSBP+bDBP [Equation 4]

In Equation 4, SBP denotes the systolic blood pressure, MAP denotes the mean arterial pressure, DBP denotes the diastolic blood pressure, and a and b are positive numbers. In addition, a relation between a and b may be defined as Equation 5 according to the characteristics of the pressure wave.

a+b=1 [Equation 5]

In Equation 5, a and b are positive numbers. In other words, a and b denote ratios of systolic and diastolic blood pressures, respectively, with respect to a whole pressure wave, and thus sum to one (1). Thus, the MAP, the DBP and the SBP may be determined by using the sphygmus wave measured in the full-pressurization mode, and a and b may be derived by using the determined blood pressures and Equations 4 and 5. In addition, Equation 4 may be rearranged as an equation for the SBP, as defined in Equation 6.

$$SBP = \frac{1}{a} MAP - \frac{b}{a} DBP \qquad [ Equation 6 ]$$

In Equation 6, SBP denotes the systolic blood pressure, MAP denotes the mean arterial pressure, DBP denotes the diastolic blood pressure, and a and b are positive numbers. Thus, a relation between a, b, n and m may be defined as Equation 7 by using Equations 6 and 3.

$$n = \frac{1}{a} , m = \frac{b}{a} \qquad [ Equation 7 ]$$

In Equation 7, a, b, n and m are positive numbers. Thus, n and m may be derived by using a and b, which are derived by using Equations 4 and 5. The relational equation may be corrected by substituting the derived n and m into Equation 3, and then stored in the memory 80. The corrected relational equation may be displayed to the user via the output unit 90.

As described above, the correction unit 71 corrects the relational equation for calculating the systolic blood pressure, thereby improving the accuracy of the blood pressure estimated in the low-pressurization mode. In other words, a relational equation according to the physical characteristics of subjects may be used, thereby improving the accuracy in blood pressure estimation in the low-pressurization mode.

Referring to FIGS. 1 and 4, the difference calculation unit 73 calculates a difference between the blood pressure measured in the full-pressurization mode and the blood pressure measured in the low-pressurization mode, and transmits the calculated difference to the output unit 90 to be displayed to the user. This is to allow the user to determine whether to correct the relational equation. The blood pressure monitoring apparatus 100 may display the necessity of correcting the relational equation to the user, such as if the difference is equal to or greater than a reference value.

In more detail, the difference calculation unit 73 calculates a difference by comparing the systolic blood pressure determined in the full-pressurization mode, and the systolic blood pressure calculated in the low-pressurization mode. If the difference is equal to or greater than a reference value, the necessity of correcting the relational equation may be displayed to the user. In one exemplary embodiment, the reference value may be 5 mmHg, but is not limited thereto.

Referring back to FIG. 1, the output unit 90 displays at least one selected from the group consisting of the blood pressure estimated by the estimation unit 71, the relational equation corrected by the correction unit 72, and the difference calculated by the difference calculation unit 73 to the user. The output unit 90 includes both a device for displaying visual information, such as a display device, a liquid crystal display (“LCD”) screen, a light-emitting diode (“LED”), or a division display device, and a device for providing auditory information, such as a speaker, in order to display information to the user. In addition, the output unit 90 may output the estimated blood pressure, the corrected relational equation, and the calculated difference to an external display device (for example, a computer monitor, or the like), in order to display at least one of these items of data to the external display device connected to the blood pressure monitoring apparatus 100.

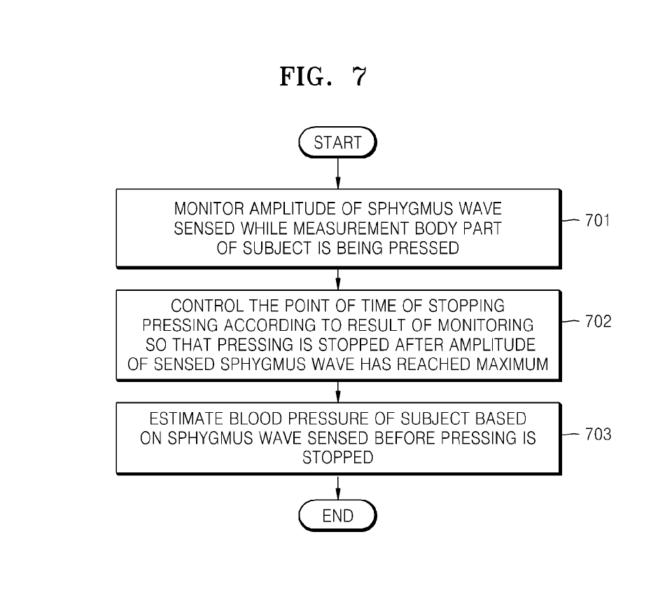

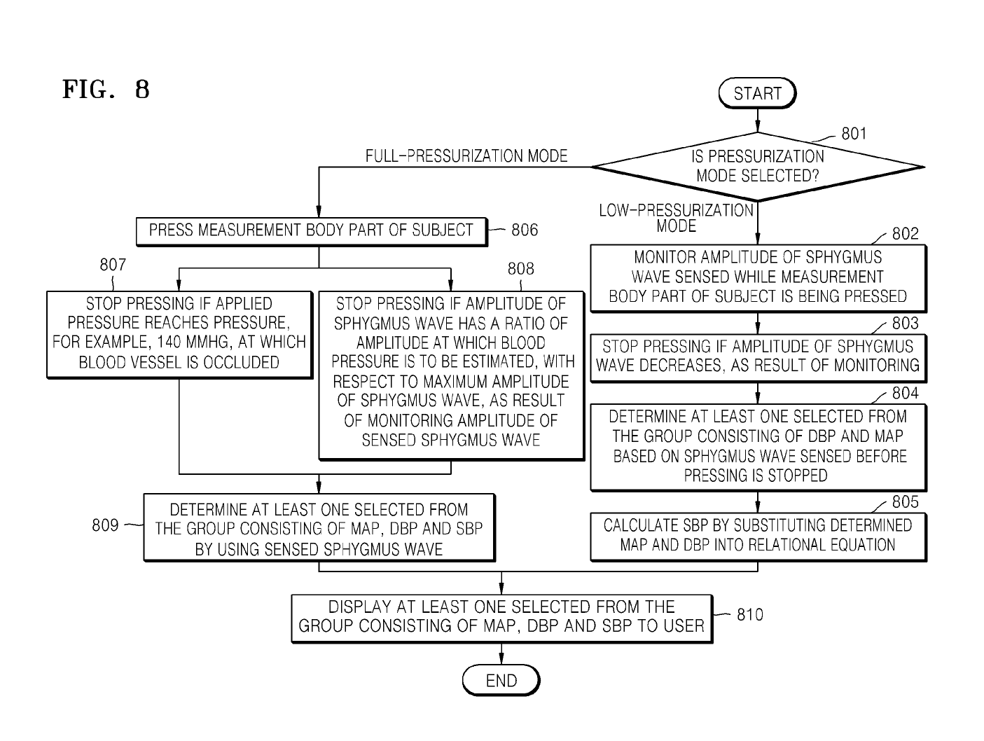

FIG. 7 is a flowchart of an exemplary embodiment of a low-pressurization blood pressure monitoring method. The blood pressure monitoring method includes operations performed sequentially in the blood pressure monitoring apparatus 100 illustrated in FIG. 1. Therefore, although not explicitly described in FIG. 7, the content described above in connection with the blood pressure monitoring apparatus 100 in FIG. 1, may also be applied to the blood pressure monitoring method according to FIG. 7.

In operation 701, the monitoring unit 50 monitors the amplitude of the sphygmus wave sensed while the measurement body part of the subject is being pressed. The pressing unit 20 presses the measurement body part of the subject, the sensing unit 30 senses the sphygmus wave at the measurement body part, and the filtering unit 40 filters the sensed sphygmus wave. The monitoring unit 50 monitors the amplitude of the filtered sphygmus wave. Herein, the measurement body part of the subject may be a wrist, a finger, an upper arm, or the like.

In operation 702, the control unit 60 controls the point of time when pressing is stopped according to the result of monitoring so that it stops after the amplitude of the sensed sphygmus wave has reached the maximum. If the low-pressurization mode is selected by using the input unit 10, the control unit 60 may control the point of time when the pressing is stopped so that it stops after the amplitude of the sensed sphygmus wave has reached the maximum. The control unit 60 may also stop the pressing performed by the pressing unit 20 if the amplitude of the sphygmus wave has decreased, as a result of monitoring.

In operation 703, the estimation unit 71 estimates blood pressure of the subject based on the sphygmus wave sensed before the pressing is stopped. The second determination unit 712 determines the mean arterial pressure and the diastolic blood pressure. The systolic blood pressure calculation unit 713 calculates the systolic blood pressure by using the determined mean arterial pressure and diastolic blood pressure. The estimated blood pressure may be displayed to the user via the output unit 90.

FIG. 8 is a flowchart of an exemplary embodiment of a blood pressure monitoring method including selecting a pressurization mode. The blood pressure monitoring method includes operations performed sequentially in the blood pressure monitoring apparatus 100 illustrated in FIG. 1. Therefore, although not explicitly described in FIG. 8, the content described above in connection with the blood pressure monitoring apparatus 100 in FIG. 1 may also be applied to the blood pressure monitoring method according to FIG. 8.

In operation 801, the input unit 10 obtains an input signal from the user. The input signal is for selecting a pressurization mode that determines a degree of pressing by the pressing unit 20. The user may select a pressurization mode that determines the degree of pressing by the pressing unit 20, by using the input unit 10. The pressurization mode may include at least one selected from the group consisting of the full-pressurization mode in which a measurement body part of a subject is pressurized until the blood vessel in the measurement body part is occluded, and the low-pressurization mode in which the measurement body part is pressurized so as not to occlude the blood vessel.

The input unit 10 may include any apparatus for obtaining the input signal from the user. In one exemplary embodiment, the input unit 10 may include a keyboard, a mouse, a touch pad, a speech recognition apparatus, or the like. If the low-pressurization mode is selected by using the input unit 10, operation 802 is performed. If the full-pressurization mode is selected, operation 806 is performed.

In operation 802 of the low-pressurization mode, the monitoring unit 50 monitors the amplitude of the sphygmus wave sensed while the measurement body part of the subject is being pressed. Operation 802 is the same as operation 701 described with reference to FIG. 7, and thus a detailed description thereof will not be provided here.

In operation 803 of the low-pressurization mode, the control unit 60 stops the pressing if the amplitude of the sphygmus wave decreases from a maximum amplitude, as a result of monitoring. The control unit 60 may stop the pressing performed by the pressing unit 20 at an instant when the amplitude of the sphygmus wave that has been continuously increasing, starts to decrease for the first time, as a result of the monitoring by the monitoring unit 50. It will be understood by one of ordinary skill in the art that the point of time when the pressing is stopped is an example, and thus is not limited thereto.

In operation 804 of the low-pressurization mode, the second determination unit 712 determines at least one selected from the group consisting of the DBP and the MAP of the subject by using the sphygmus wave sensed before the pressing is stopped. A method of determining the DBP and the MAP has been described above with reference to FIG. 4, and thus a detailed description thereof will not be provided here.

In operation 805 of the low-pressurization mode, the systolic blood pressure calculation unit 713 calculates the SBP by substituting the MAP and the DBP obtained from the second determination unit 712 into the relational equation representing the correlation between the MAP, the DBP and the SBP.

If the full-pressurization mode is selected in operation 801, the pressing unit 20 presses the measurement body part of the subject in operation 806. The point of time when the pressing is stopped is determined according to operation 807 or operation 808. One of the methods of pressing the measurement body part for the full-pressurization mode may be stored as a default according to a usage environment or may be selected by the user via the input unit 10.

In operation 807 of the full-pressurization mode, the control unit 60 stops the pressing performed by the pressing unit 20 if the pressure applied by the pressing unit 20 increases and reaches 140 mmHg, which is known as a pressure at which the blood vessel is occluded.

In operation 808 of the full-pressurization mode, the control unit 60 stops the pressing performed by the pressing unit 20 by using the result of the monitoring performed by the monitoring unit, e.g., if the amplitude of the sphygmus wave has a ratio at which blood pressure is to be estimated, with respect to the maximum amplitude of the sphygmus wave. Herein, the ratio at which blood pressure is to be estimated refers to a characteristic ratio generally used to calculate the SBP, and may be, for example, about 40% of the maximum amplitude of the sphygmus wave.

In operation 809 of the full-pressurization mode, the first determination unit 711 determines at least one selected from the group consisting of the MAP, the DBP and the SBP, by using the sensed sphygmus wave. Methods of determining the MAP, the DBP and the SBP have been described above with reference to FIG. 4, and thus a detailed description thereof will not be provided here.

In operation 810, the output unit 90 may display at least one selected from the group consisting of the MAP, the DBP and the SBP from operation 805 and/or operation 809, to the user.

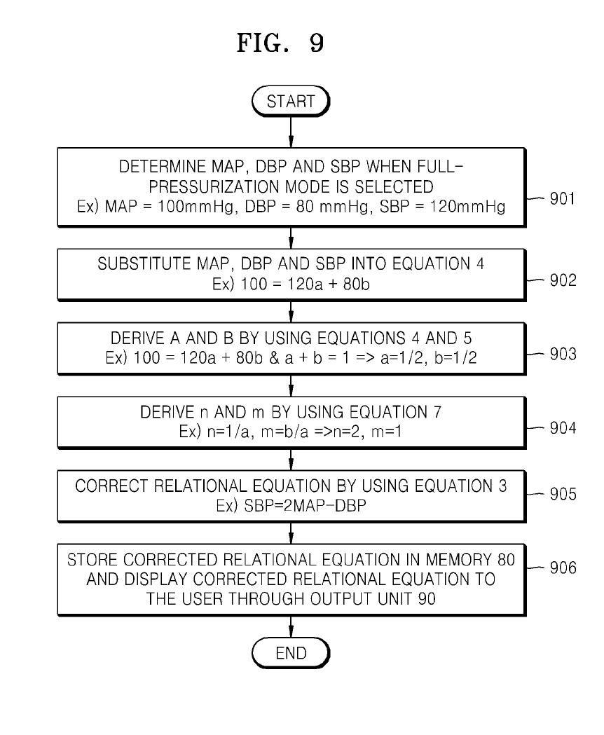

FIG. 9 is a flowchart of an exemplary embodiment of a method of correcting the relational equation.

In operation 901, when the full-pressurization mode is selected through the input unit 10, the first determination unit 711 determines the MAP, the DBP and the SBP by using the sphygmus wave sensed at the measurement body part of the subject. Methods of determining the MAP, the DBP and the SBP have been described above with reference to FIG. 4, and thus a detailed description thereof will not be provided here. In one exemplary embodiment, the MAP, the DBP and the SBP may be determined as 100 mmHg, 80 mmHg, and 120 mmHg, respectively.

In operation 902, the MAP, the DBP and the SBP determined in operation 901 are substituted into Equation 4. Equation 4 defines the correlation between the MAP, the DBP and the SBP. Thus, when the MAP, the DBP and the SBP determined in operation 901 are substituted into Equation 4, Equation 8 is defined as follows.

100=120a+80b [Equation 8]

In operation 903, a and b are derived by using Equations 4 and 5. A method of deriving a and b by using Equations 4 and 5 is obvious to one of ordinary skill in the art, and thus a detailed description thereof will not be provided here. In the illustrated embodiment, a and b derived by using Equation 8, defined in operation 902, and Equation 5, may be ½ and ½, respectively.

In operation 904, n and m are derived by using Equation 7. In the illustrated embodiment, n and m derived by using Equation 7 may be 2 and 1, respectively.

In operation 905, the relational equation is corrected by substituting n and m into Equation 3. Thus, the corrected relational equation may be defined as Equation 9.

SBP=2MAP−DBP [Equation 9]

The relational equation previously stored in the memory 80 may be updated with the corrected relational equation in operation 906. In addition, the corrected relational equation may be displayed to the user through the output unit 90.

The method of correcting the relational equation as described in FIG. 9 may be automatically performed in the blood pressure monitoring apparatus 100 according to a difference calculated by the difference calculation unit 73, or may be arbitrarily performed by the user. In one exemplary embodiment, if the difference calculated by the difference calculation unit 73 is equal to or greater than a reference value, the relational equation may be corrected as described with reference to FIG. 9.

According to the blood pressure monitoring method described above, blood pressure of a subject may be measured in a low-pressurization mode under the control of the control unit 60, which controls the point of time when the pressing is stopped. Thus, the user may feel less inconvenienced and may experience less side effects caused by the occlusion of blood vessels. In addition, a user who frequently measures blood pressure may feel much less pain during blood pressure measurement. Since less pressure is applied in the low-pressurization mode than in the full-pressurization mode to measure blood pressure, the pressurizing duration becomes shorter, so that the overall blood pressure measurement duration may be reduced.

In the blood pressure monitoring method described above, a pressurization mode may be selected from among multiple pressurization modes, by using the input unit. Consequently, the accuracy in blood pressure estimation in the low-pressurization mode is improved by using the full-pressurization mode. Thus, blood pressure may be conveniently and accurately measured by using the blood pressure monitoring apparatus 100 configured to execute both the low-pressurization mode and the full-pressurization mode.

As described above, according to the one or more of the above aspects of the invention, blood pressure of a subject may be measured in a low-pressurization mode in which the blood vessels of the subject are not occluded. Thus, the subject may measure blood pressure without inconvenience and side effects caused by the repeated occlusion of the blood vessels. In addition, the accuracy in blood pressure estimation in the low-pressurization mode may be improved by using blood pressures of the subject measured in a full-pressurization mode in which pressure that is strong enough to occlude the blood vessels is applied. In other words, it is possible to manufacture a blood pressure monitoring apparatus that allows a user to select between multiple pressurization modes, thus enabling convenient, accurate blood pressure estimation.

The exemplary embodiments of the blood pressure monitoring method detailed above, may be written as computer programs and may be implemented in general-use digital computers that execute the programs using a computer readable recording medium. Data used in the above-described examples may be recorded on a medium in various means. The computer readable recording medium include magnetic storage media (e.g., read-only memory (“ROM”), floppy disks, hard disks, etc.) and optical recording media (e.g., compact discs, read-only memory (“CD-ROMs”), or digital versatile discs (“DVDs”)).

While this invention has been particularly shown and described with reference to exemplary embodiments thereof, it will be understood by those of ordinary skill in the art that various changes in form and details may be made therein without departing from the spirit and scope of the invention as defined by the appended claims. The exemplary embodiment should be considered in descriptive sense only and not for purposes of limitation. Therefore, the scope of the invention is defined not by the detailed description of the invention but by the appended claims, and all differences within the scope will be construed as being included in the present invention.

What is claimed is:

1. A blood pressure monitoring apparatus comprising:
a pressing unit which presses a measurement body part of a subject;
a sensing unit which senses a sphygmus wave at the measurement body part;
a control unit which stops the pressing performed by the pressing unit based on an amplitude of the sensed sphygmus wave; and
an estimation unit which estimates a blood pressure of the subject, based on the sphygmus wave sensed before the pressing performed by the pressing unit is stopped.

2. The blood pressure monitoring apparatus of claim 1, wherein the control unit stops the pressing performed by the pressing unit after the amplitude of the sensed sphygmus wave has reached a maximum.

3. The blood pressure monitoring apparatus of claim 1, further comprising a monitoring unit which monitors the amplitude of the sensed sphygmus wave,
wherein the control unit stops the pressing performed by the pressing unit when the amplitude of the sensed sphygmus wave decreases, as a result of the monitoring of the monitoring unit.

4. The blood pressure monitoring apparatus of claim 1, wherein the estimation unit comprises a determination unit which determines a first pressure having a maximum amplitude of the sensed sphygmus wave and a second pressure having a ratio of amplitude at which blood pressure is to be estimated, with respect to the maximum amplitude of the sensed sphygmus wave, and
the estimation unit estimates the determined first and second pressures from the determination unit, as the blood pressure of the subject.

5. The blood pressure monitoring apparatus of claim 4, wherein the estimation unit further comprises a calculation unit which calculates a systolic blood pressure based on the first pressure and the second pressure determined by the determination unit, and
the estimation unit estimates the calculated systolic blood pressure from the calculation unit, as the blood pressure of the subject.

6. The blood pressure monitoring apparatus of claim 5, wherein the calculation unit calculates the blood pressure of the subject by using a relational equation representing a correlation between the first pressure, the second pressure, and the systolic blood pressure.

7. The blood pressure monitoring apparatus of claim 1, wherein the control unit stops the pressing performed by the pressing unit when a pressure applied by the pressing unit reaches a pressure at which blood vessels are occluded.

8. The blood pressure monitoring apparatus of claim 7, further comprising an input unit which obtains an input signal for selecting a pressurization mode which determines a degree of pressing by the pressing unit,
wherein the control unit determines a point of time when the pressing performed by the pressing unit is stopped according to the input signal.

9. The blood pressure monitoring apparatus of claim 7, wherein the estimation unit further comprises a determination unit which determines at least one selected from the group consisting of mean arterial pressure, diastolic blood pressure and systolic blood pressure, by using the sphygmus wave sensed while the measurement body part is being pressed until the pressure applied by the pressing unit reaches the pressure level at which the blood vessels are occluded, and
the estimation unit estimates the determined at least one blood pressure from the determination unit, as the blood pressure of the subject.

10. The blood pressure monitoring apparatus of claim 9, further comprising a correction unit which corrects a relational equation representing a correlation between a pressure having a maximum amplitude of the sensed sphygmus wave and a pressure having a ratio of amplitude at which blood pressure is to be estimated, with respect to the maximum amplitude of the sensed sphygmus wave, by using the determined at least one blood pressure from the determination unit.

11. The blood pressure monitoring apparatus of claim 9, further comprising a difference calculation unit which calculates a difference between blood pressures measured by using the sphygmus wave sensed before the pressing is stopped,
wherein the blood pressures from which the difference is calculated are measured, respectively, while the pressing is stopped based on an amplitude of the sensed sphygmus wave, and while pressing is stopped after the pressure applied by the pressing unit has reached a pressure level at which blood vessels are occluded.

12. The blood pressure monitoring apparatus of claim 1, wherein the pressure applied to the measurement body part of the subject by the pressing unit is continuously increased until the pressing performed by the pressing unit is stopped by the control unit.

13. A blood pressure monitoring method comprising:
monitoring an amplitude of a sphygmus wave sensed while a measurement body part of a subject is being pressed;
controlling a point of time of stopping the pressing according to a result of the monitoring, and stopping the pressing after the amplitude of the sensed sphygmus wave has reached a maximum, and
estimating a blood pressure of the subject based on the sphygmus wave sensed before the pressing performed by the pressing unit is stopped.

14. The blood pressure monitoring method of claim 13, wherein, in the controlling, the point of time when the pressing is stopped is controlled according to the result of the monitoring, so that pressing stops when the amplitude of the sensed sphygmus wave decreases.

15. The blood pressure monitoring method of claim 13, wherein the estimating a blood pressure further comprises calculating a systolic blood pressure based on a first pressure having a maximum amplitude of the sensed sphygmus wave, and a second pressure having a ratio of amplitude at which blood pressure is to be estimated, with respect to the maximum amplitude of the sensed sphygmus wave,
wherein the calculated systolic blood pressure is estimated as a systolic blood pressure of the subject.

16. The blood pressure monitoring method of claim 13, wherein the controlling is performed so that the pressing is stopped when a pressure applied for the pressing has reached a pressure at which blood vessels are occluded.

17. The blood pressure monitoring method of claim 16, further comprising obtaining an input signal for selecting a pressurization mode which determines a degree of pressing,
wherein the controlling a point of time of stopping the pressing is performed according to the input signal.

18. The blood pressure monitoring method of claim 17, wherein the estimating a blood pressure further comprises:
determining at least one selected from the group consisting of mean arterial pressure, diastolic blood pressure and systolic blood pressure of the subject by using the sphygmus wave sensed while the measurement body part is being pressed until the pressure applied for the pressing reaches the pressure level at which the blood vessels are occluded, and
correcting a relational equation representing a correlation between a pressure having the maximum amplitude of the sensed sphygmus wave and a pressure having a ratio of amplitude at which blood pressure is to be estimated, with respect to the maximum amplitude of the sensed sphygmus wave, by using the determined at least one blood pressure.

19. The blood pressure monitoring method of claim 17, further comprising calculating a difference between blood pressures measured by using the sphygmus wave sensed before the pressing is stopped,
wherein the blood pressures from which the difference is calculated are measured, respectively, while pressing is stopped based on the amplitude of the sphygmus wave, and while pressing is stopped after the pressure applied to the measurement body part has reached a pressure level at which blood vessels are occluded.

20. A computer readable recording medium having embodied thereon a program for executing the method of claim 13.
